(19) United States
(12) Patent Application Publication
(10) Pub. No.: US 2022/0011282 A1
(43) Pub. Date: Jan. 13, 2022

(54) Method for Electrochemical Gas Sensor Diagnostics

(71) Applicant: Aeroqual Ltd., Auckland (NZ)

(72) Inventors: Geoffrey Stephen Henshaw, Auckland (NZ); Anna Kate Farquhar, Auckland (NZ); David Edward Williams, Kerikeri (NZ)

(21) Appl. No.: 17/371,089

(22) Filed: Jul. 8, 2021

Related U.S. Application Data
(60) Provisional application No. 63/049,168, filed on Jul. 8, 2020.

(51) Int. Cl. G01N 33/00

(52) U.S. Cl. CPC G01N 33/0006

(57) ABSTRACT

Baseline noise in electrochemical sensors is useful. Reduction in baseline noise over a period of time when an electrochemical sensor is exposed to sudden changes in environmental conditions provides indication that an electrochemical sensor should be replaced. Stop changes in sensitivity confirm that test. Drops in baseline noise during high winds predict weather events. Frequency of electrochemical sensor noise is used to detect ambient sound waves.

This application claims the benefit of U.S. Provisional Application No. 63/049,168 filed Jul. 8, 2020, which is hereby incorporated by reference in its entirety as if fully set forth herein.

### BACKGROUND OF THE INVENTION

The present invention describes a method for diagnosing electrochemical gas sensor condition using the sensors response to environmental noise.

Electrochemical gas sensors have 2-3 sensor electrodes, including a working (sensing) electrode, surrounded by an electrolyte. The electrode/electrolyte system is enclosed in a sensor housing that is separated from the external environment by a gas permeable membrane. In an electrochemical gas sensor, the target gas diffuses through the membrane and electrolyte, to the working electrode where it is either reduced or oxidized, generating an electrical current. Gas sensing electrodes are designed such that the electrochemical reaction kinetics are very fast, hence the rate determining step is mass transport of the target gas to the electrode. Typically, the rate determining step is diffusion through the gas permeable membrane. This is achieved by designing appropriate electrode catalysts and defining an appropriate electrode potential for the target gas. Having a diffusion limited device ensures a linear relationship between the gas concentration and the current generated at the working electrode. Degradation of the sensor electrodes can slow down the kinetics of the working electrode reaction, hence the sensor will begin to operate in a mixed kinetic/diffusion mode, leading to sensor drift and non-linearity. At this point the sensor readings become unreliable and the GSE sensor should likely be replaced. Methods to diagnose sensor condition are therefore essential to ensure reliable gas monitoring.

U.S. Pat. No. 6,049,283 (EP0841563B1) and U.S. Pat. No. 8,543,340 describe a method for alerting a sensor fault condition by monitoring the output signal of the amplifying circuit, and triggering an alarm when the signal noise (standard deviation, rms, or variance) falls below a threshold value, or is trending downward for an extended period of time. US20060042960A1 uses the stochastic noise of a CO sensor to calculate a gain parameter based on predetermined reference data to correct for sensitivity loss. U.S. Pat. Nos. 6,428,684B1, 558,752, and WO99/22232 confirm a sensor is operating in a diffusion limited regime by changing the applied potential of the working electrode, and plotting potential vs. current, as in a diffusion limited electrochemical process, current is independent of the applied potential. U.S. Pat. No. 7,090,755B2 and EP2327981B1 confirm a sensor is diffusion limited by operating the sensor with reduced reaction capacity thereby allowing gas to accumulate, then increasing the activity and measuring the transient response. These methods require a controlled supply of the target gas. Electrochemical methods including potential step techniques (US20190170679A1, DE4445947C2, U.S. Pat. No. 8,160,834B2) or impedance spectroscopy (WO2000014523A2, US20110199094A1) are used to calculate properties including capacitance and resistance as an indication of sensor health. U.S. Pat. No. 6,629,444 exploits the response of am electrochemical gas sensor to a sudden change in water vapor pressure and measures the transient current response. If the current spike exceeds a predetermined threshold, the sensor is deemed operational. This diagnostic method requires a specialist set up.

Needs exist for improved sensor condition monitoring and diagnostics.

### SUMMARY OF THE INVENTION

It is the aim of the present invention to use the response of an electrochemical sensor to environmental changes to determine the health of the sensor and establish when a sensor requires replacing.

It has been determined that the baseline electrical output signal (current) of an electrochemical sensor will fluctuate when the sensor is exposed to sudden changes in environmental conditions, including dew point, relative humidity, water vapor pressure, temperature, pressure, and wind speed. Rapid fluctuations in these parameters cause noise in the electrochemical sensor baseline.

Furthermore, after extended use, electrochemical sensors undergo a step change in sensitivity, manifesting as an irreversible decrease in sensitivity. It is believed that this corresponds to the transition from diffusion limited behaviour to a mixed kinetic/diffusion regime, resulting from degradation of the working electrode catalyst and surface area. At this time, the sensor should be replaced. The irreversible drop in sensitivity coincides with a decrease in the sensors response to fluctuations in environmental conditions and therefore a decrease in statistical measures of the electrical output signal noise of the electrochemical sensor. This decrease in noise in the can therefore be used to determine when a sensor needs replacing.

It has also been noted that during periods of high winds, i.e. anomalous weather events, the electrical output signal noise of the sensor drops significantly. The present invention uses this drop in baseline noise to predict weather events.

A further embodiment of this invention is to use an electrochemical sensors response to environmental conditions in neural network applications to estimate the sensor response. Finally, this invention uses the frequency of the electrical output signal noise of an electrochemical gas sensor to detect ambient soundwaves.

These and further and other objects and features of the invention are apparent in the disclosure, which includes the above and ongoing written specification, with the claims and the drawings.

### BRIEF DESCRIPTION OF THE DRAWINGS

FIG. 1 shows how a baseline current of an electrochemical sensor responds to a sudden decrease then an increase in the dewpoint of its environment.

FIG. 2 shows at the top a concentration of a gas and at the bottom a reference concentration in dashes and dewpoint in solid line over the same period of time.

FIG. 3 shows stops in a process for using sensors sensor noise due to environmental fluctuations as a diagnostic.

FIGS. 4A and 4B show a sensitivity of an electrochemical sensor in mV per ppm over 1 year and a ratio of electrical output of the electrochemical sensor to the standard deviation of dewpoint over the same 1 year period.

FIG. 5A shows an electrochemical sensor output compared to wind speed (dash lines).

FIG. 5B shows electrochemical sensor output compared to wind direction during the same period of weather events.

FIG. 6 shows stops in using baseline noise to detect nearby sounds.

FIG. 7 shows baseline current in the absence and presence of a sound wave a fixed frequency.

### DETAILED DESCRIPTION

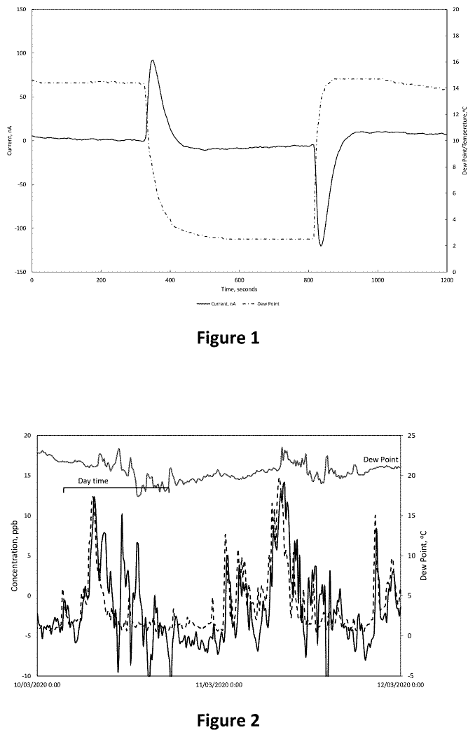

The baseline current of an electrochemical gas sensor responds to changes in environmental conditions. The baseline current is most likely due to oxygen reduction and oxidation of the working electrode. The diffusion limits that control the electrochemical reaction of the target gas allowing the correct operation of an electrochemical gas sensor are not necessarily true for the background current reactions, and so the background current depends on the working electrode composition and area, and the electrolyte composition. Changes in the relative humidity/dew point/water vapor pressure, temperature, air pressure, and windspeed causes a transient current spike in the baseline/background current, most likely due to a change in the rate of the baseline current reaction at the working electrode surface. This could be caused by a fluctuation in the working electrode area or a fluctuation in the composition of the sensor electrolyte due to the environmental change. FIG. 1 shows how the current response of an electrochemical gas sensor responds to a sudden decrease then increase in the dew point. Transient current responses are seen with an increase or decrease of dew point/relative humidity/water vapor pressure, temperature, pressure, and windspeed. The magnitude of the current transient depends on the magnitude and direction of the environmental fluctuation.

FIG. 1 shows a diagnostic change baseline current of an electrochemical sensor as the dew point is decreased and then increased.

The fluctuations in current due to fluctuations in environmental conditions manifest as baseline noise in the electrical signal output of an electrochemical sensor. FIG. 2 shows the concentration output of an electrochemical gas sensor operated outside over a 2-day period, and the dew point over the same time-period. Periods of high dew point noise (during the day) coincide with rapid fluctuations in the baseline current (baseline noise) that cannot be ascribed to target gas (reference data shown as black dashed line). As the baseline current depends on the condition of the working electrode, it therefore follows that a shift in how the baseline current responds to fluctuations in an environmental parameter (dew point, temperature, pressure, or windspeed) is indicative of sensor health.

FIG. 2 shows a reported concentration of an electrochemical gas sensor operating outside and reference concentration over the same time-period (dashed black), and dew point (dark grey).

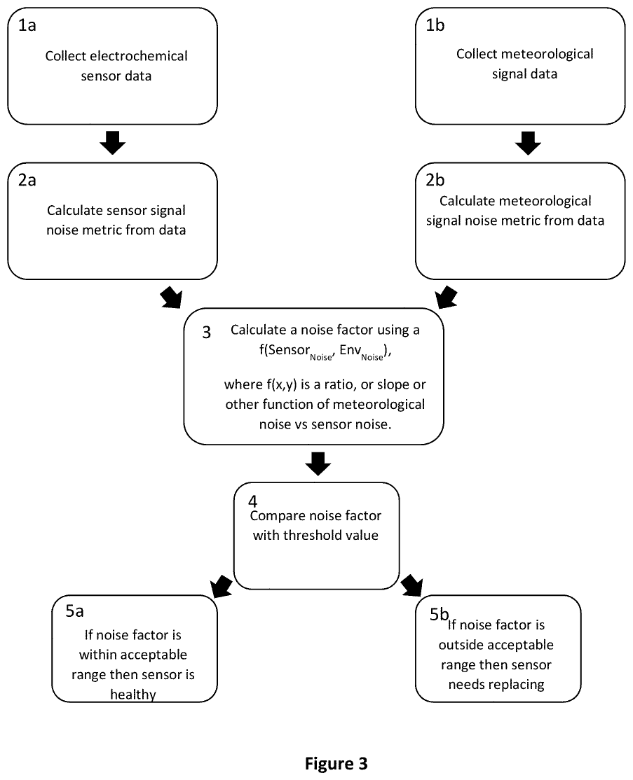

FIG. 3 shows a flow chart outlining process for using noise in sensor response due to environmental fluctuations as a diagnostic.

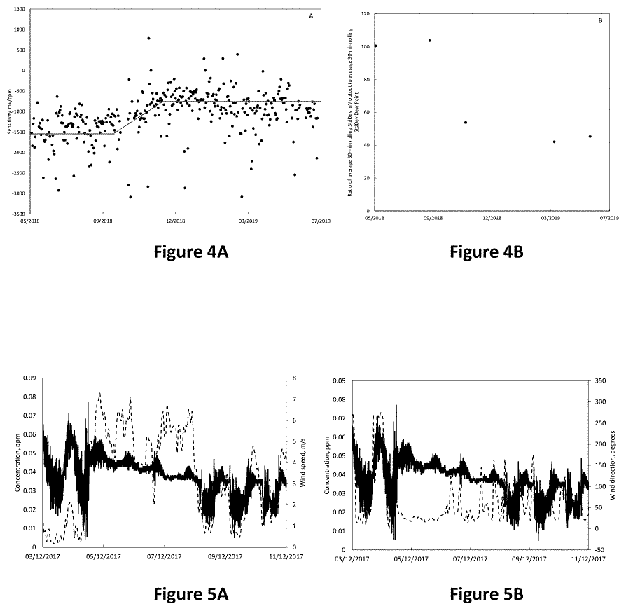

FIG. 3 illustrates the process for using noise in the sensor output due to an environmental fluctuation as a diagnostic tool. Box 1A and 1B describe collecting the time series electrical output data for the electrochemical sensor and the environmental conditions over the same time-period, respectively. In the example described by FIG. 4, the electrical output signal is the mV output (concentration or sensor current could also be used), and the environmental condition is dew point (relative humidity, water vapor pressure, temperature, air pressure, or windspeed could also be used). In Box 2A and 2B the same noise metric is calculated for the output signal and environmental condition. For the example in FIG. 4 the noise metric is standard deviation (RMS, variance, and other common statistical measures of noise could also be used). Box 3 calculates a noise factor using a function of the sensor noise and environmental noise (f(Sensor_{Noise}, Env_{Noise})), where for the example in FIG. 4 the function is the ratio. The noise factor could also be the slope of an environmental parameter vs. sensor output plot, or any other common mathematical function. In box 5 the noise factor is compared to a predetermine threshold. The sensor is deemed to be healthy is the noise factor is greater than the threshold, or the sensor needs replacing if the factor is below the threshold. In the example in FIG. 4, the noise factor decrease is attributed to electrode degradation causing a loss of sensitivity and a transition from diffusion limited, to mixed diffusion/kinetic limited behaviour.

FIG. 4A shows sensitivity of a GSE electrochemical sensor in mV per ppm over 1-year period. FIG. 4B shows the ratio of the standard deviation of electrical output signal of the sensor to the standard deviation of dew point over the same 1-year time-period.

A further embodiment of this invention defines the noise factor as the ratio between the sensor output noise metric during the day and at night. During the day, the environmental fluctuations are significant, hence sensor noise level is higher compared to at night. The noise metric is defined as the standard deviation, or the variance, or the rms, or other common statistical measure of noise of the electrical output signal of the sensor. A decrease in the noise factor below a predetermined threshold indicates the sensor needs replacing. In the second part of this invention, the output signal of the sensor is used to predict anomalous weather events. FIG. 5 shows the sensor output over a 1-month time-period for an electrochemical gas sensor operating in California. A period of significantly lower than average noise occurs during high speed, northeasterly winds (Santa Ana winds). A reduction in the noise factor is used to predict an extreme weather event. This example uses a sudden, reversible reduction in the sensor output standard deviation to predict an extreme weather event. A further embodiment is to use the change in variance or rms or other statistical measure of noise of the sensor output signal to predict an extreme weather event.

FIG. 5A shows electrochemical sensor output and windspeed (dashed) during a weather event. FIG. 5B shows electrochemical sensor output and wind direction (dashed) during the same weather event.

In the third part of this invention, methods that use noise in neural network applications are described. The electrochemical gas sensor responds to changes in dew point/relative humidity/water vapor pressure, temperature, pressure, and windspeed in a predictable and reproducible manner. It therefore follows that neural networks can be used to estimate the baseline concentration using the values and standard deviation of the dew point, or relative humidity or water vapor pressure. Further embodiments use the values and the standard deviation of the temperature, or air pressure, or wind speed in neural networks to estimate the baseline concentration. The values and rms of, or values and variance of the dew point, or relative humidity, or water vapor pressure, or temperature, or pressure, or wind speed, are also used in neural network applications to predict baseline concentration. Neural networks based on the above parameters are also used to estimate the concentration of the target gas.

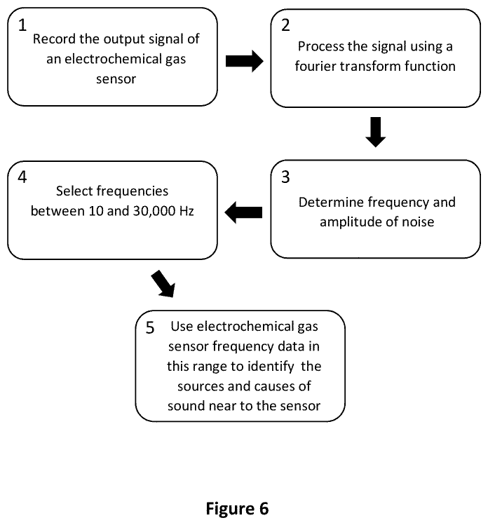

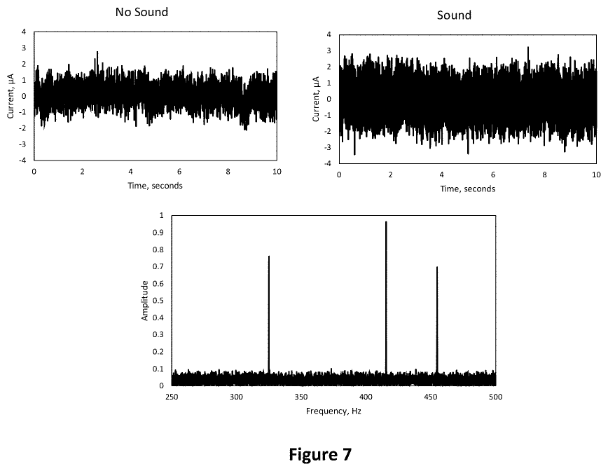

FIG. 6 outlines the final part of this invention, where baseline noise is used to detect sound. In box 1 the output signal of the electrochemical sensor is recorded. For the example the baseline current is recorded using a potentiostat at 1 ms (1000 Hz) intervals. A signal processing step, for example Fourier transform (box 2) is applied to the data. The frequency and amplitude of the noise is then used to determine ambient sound frequencies surrounding the sensor, where sound is defined as baseline noise with a frequency of 10-10,000 Hz. Using known frequencies of common sounds, the data is used to diagnose sources of noise pollution and is used to map the landscape around the sensor. As an example, the peak at 450 Hz can be ascribed to the low rumble of a train (box 5).

FIG. 6 is a flow chart outlining steps to use electrochemical sensor baseline noise to detect ambient sounds.

FIG. 7 shows baseline current in the absence (top left) and presence (top right) of a soundwave of fixed frequency. Bottom—Fourier transform of baseline current of electrochemical sensor operated in the presence of a mixture of soundwaves.

While the invention has been described with reference to specific embodiments, modifications and variations of the invention may be constructed without departing from the scope of the invention, which is defined in the following claims.

1-18. (canceled)

19. A method to diagnose the health of an electrochemical gas sensor by:
collecting time series data of the output signal of an electrochemical gas sensor and calculating the signal noise,
collecting time series data of output signal of a meteorological sensor over the same time-period and calculating the meteorological signal noise,
calculating a noise factor which is a mathematical function of the electrochemical gas sensor noise and the meteorological sensor noise and using this to determine when the sensor needs replacing.

20. The method according to claim 19, where the noise factor is the ratio or slope of the sensor noise to the meteorological sensor noise.

21. The method according to claim 19, where a metric used to determine the electrochemical gas sensor signal noise is standard deviation, or variance, or root mean square.

22. The method according to claim 19, where the output signal of the electrochemical gas sensor is voltage, current, or concentration.

23. The method according to claim 19, where the meteorological sensor measures dew point or relative humidity or water vapor pressure or temperature or wind speed or wind direction or pressure.

24. The method according to claim 19, where the electrochemical gas sensor is deemed to need replacing when the noise factor falls above or below a predetermined threshold.

25. The method according to claim 19 to diagnose the health of an electrochemical gas sensor by:
collecting the time series data of the output signal of the electrochemical gas sensor and calculating the signal noise during the day and at night,
calculating a noise factor which is a mathematical function of the electrochemical gas sensor signal noise during the day and the electrochemical gas sensor signal noise during the night and using this to determine when the sensor needs replacing.

26. The method according to claim 25, where mathematical function is the ratio between the gas sensor signal noise during the day and the signal noise at night.

27. The method according to claim 25, where a metric used to determine the electrochemical gas sensor signal noise is standard deviation, or variance, or root mean square.

28. The method according to claim 25, where the sensor is deemed to need replacing when the noise factor is above or below a predetermined threshold.

29. A method to identify a weather event by using the electrical output signal noise of an electrochemical gas sensor.

30. The method according to claim 29, where the metric to determine the noise is standard deviation, or root mean square, or variance.

31. The method according to claim 29, where a weather event is defined as a time-period where there is a sudden, reversible decrease or increase in noise of the electrochemical gas sensor output.

32. A method to estimate baseline current of electrochemical gas sensors using the values of dew point (relative humidity/water vapor pressure), temperature, pressure or wind speed, and a statistical measure of their noise in a neural network.

33. The method according to claim 32, where the statistical measure of noise is standard deviation, or root mean square, or variance.

34. A method comprising detecting ambient sound using the frequency of the baseline response of an electrochemical gas sensor by:
collecting the output signal of the electrochemical sensor at a predetermined frequency,
using signal processing techniques to determine the frequency and amplitude of the signal noise,
using the electrochemical sensor signal between frequency 10 and 30,000 Hz to identify sources of sound near the sensor.
